(19) United States
(12) Patent Application Publication
(10) Pub. No.: US 2001/0005711 A1
(43) Pub. Date: Jun. 28, 2001

(54) Perfume compositions comprising 4,8-dimethyl-3,7-nonadien-2-one

(76) Inventors: Stefan Lambrecht, Holzminden (DE); Erich Dilk, Holzminden (DE); Peter Worner, Holzminden (DE)

Correspondence Address: Patent Department, Bayer Corporation, 100 Bayer Road, Pittsburgh, PA, 15205-9741, US

(21) Appl. No.: 09/736,599

(22) Filed: Dec. 13, 2000

(30) Foreign Application Priority Data
Dec. 21, 1999 (DE) 19961598.5

(51) Int. Cl. A61K 7/46

(52) U.S. Cl. 512/26

(57) ABSTRACT

Perfume compositions, which comprise 4,8-dimethyl-3,7-nonadien-2-one can be used in fine perfumery and for the perfuming of industrial products.

### FIELD OF THE INVENTION

The invention relates to perfume compositions, which comprise 4,8-dimethyl-3,7-nonadien-2-one.

### BACKGROUND OF THE INVENTION

Despite a large number of existing fragrances, there continues to be a need in the perfume industry for novel fragrances which, beyond their olfactory properties, have additional positive secondary properties, such as, for example, better stability, better odor strength and better substantivity on fabric, and better staying power.

4,8-Dimethyl-3,7-nonadien-2-one is known per se (DE A 22 56 347). The olfactory properties have been described as fruity, fresh, acetate, rose oxide, aqueous grapefruit, melon, fresh, bergamot and rosemary (Flavour Fragrance J., 1996, 11, 15-23; Indian Perfum., 1983, 27, 112-18).

### SUMMARY OF THE INVENTION

The present invention relates to perfume compositions comprising E- and/or Z-4,8-dimethyl-3,7-nonadien-2-one of the formulae

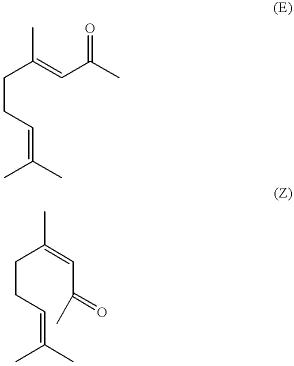

as an individual isomer or as a mixture.

These compounds are particularly suitable as fragrances, which can be used in perfumings.

The compound according to the present invention is generally in the form of a mixture of two. It is surprising that E- and/or Z-4,8-dimethyl-3,7-nonadien-2-one in fragrance compositions not only has the known olfactory properties, but is intensified in the composition and thus can be perceived even in extremely small amounts.

It is further surprising that these compositions have higher color stability than compositions with comparable odor properties. If E- and/or Z-4,8-dimethyl-3,7-nonadien-2-one is used instead of other similar fragrances, such as citronellal or citral, no discolorations of any kind arise in the perfume composition. Color changes in the perfume composition make them less acceptable. It is, therefore, an aim of the perfume industry to increase the color stability of perfume compositions.

In addition, 4,8-dimethyl-3,7-nonadien-2-one has the surprising property of long-lastingness, of freshness boosting and of harmonizing the sensory impression.

Because of these particular properties, mixtures of E- and/or Z-4,8-dimethyl-3,7-nonadien-2-one are highly suitable for use in perfume compositions.

### DETAILED DESCRIPTION OF THE INVENTION

The inclusion of E- and/or Z-4,8-dimethyl-3,7-nonadien-2-one, even in low concentrations in the resulting perfume compositions, surprisingly achieves radiantly fresh, rounded odor notes, the overall olfactory impression being harmonized to a striking degree, the cosmetic-floral character being perceptibly increased and the fixing, i.e., the substantivity of the perfume oil, being significantly improved. Examples of fragrances which can be combined according to the present invention with E- and/or Z-4,8-dimethyl-3,7-nonadien-2-one to give perfume compositions can be found, for example, in S. Arctander, Perfume and Flavor Materials, Vol. I and II, Montclair, N.J., 1969, published privately or K. Bauer, D. Garbe and H. Surburg, Common Fragrance and Flavor Materials, 3^{rd}. Ed., Wiley-VCH, Weinheim 1997.

These fragrances include:

Extracts from natural raw materials, such as, essential oils, concretes, absolutes, resins, resinoids, balsams, tinctures, such as, for example, ambergris tincture; amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; corianda oil; costus root oil; cumin oil; cypress oil; Davana oil; dill herb oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gudun balsam oil; helichrysum absolute; helichrysum oil; ginger oil; iris root absolute; iris root oil; jasmine absolute; calamus oil; camomile blue oil; camomile Roman oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lavage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtenol; clove leaf oil; clove flower oil; neroli oil; olibanum absolute; olibanum oil; opopanax oil; orange-flower absolute; orange oil; origanum oil; palmarosa oil; patchouli oil; perilla oil; peruvian balsam oil; parsley leaf oil; parsley seed oil; petitgrain oil; peppermint oil; pepper oil; pimenta oil; pine oil; pennyroyal oil; rose absolute; rosewood oil; rose oil; rosemary oil; dalmation sage oil; Spanish sage oil; sandalwood oil; celery seed oil; spike lavender oil; Japanese anise oil; styrax oil; tagetes oil; fir needle oil; teatree oil; turpentine oil; thyme oil; Tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom;

individual fragrances from the group of hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;

of aliphatic alcohols, such as, for example, hexanol; octanol; 3-octanol; 2,6-dimethylheptanol; 2-methylheptanol, 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxy-octan-2-ol; 9-decenol; 10-undecenol; 4-methyl-3-decen-5-ol; of aliphatic aldehydes and acetals thereof, such as, for example, hexanal; heptanal; octanal; nonanal; decanal; undecanal; dodecanal; tridecanal; 2-methyloctanal; 2-methylnonanal; (E)-2-hexenal; (Z)-4-heptenal; E- and Z-4,8-dimethyl-3,7-nonadien-2-ol; 2,6-dimethyl-5-heptenal 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyl oxyacetaldehyde;

of aliphatic ketones and oximes thereof, such as, for example, 2-heptanone; 2-octanone; 3-octanone; 2-nonanone; 5-methyl-3-heptanone; 5-methyl-3-heptanone oxime; 2,4,4,7-tetramethyl-6-octen-3-one; of aliphatic sulphur-containing compounds, such as, for example, 3-methylthiohexanol; 3-methylthiohexyl acetate; 3-mercaptohexanol; 3-mercaptohexyl acetate; 3-mercaptohexyl butyrate; 3-acetyl-thiohexyl acetate; 1-menthene-8-thiol;

of aliphatic nitriles, such as, for example, 2-nonenenitrile; 2-tridecenenitrile; 2,12-tridecadienenitrile; 3,7-dimethyl-2,6-octadienenitrile; 3,7-dimethyl-6-octene-nitrile;

of aliphatic carboxylic acids and esters thereof, such as, for example, (E)- and (Z)-3-hexenyl formate; ethyl acetoacetate; isoamyl acetate; hexyl acetate; 3,5,5-trimethyl-hexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate, isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl 2-isoamyloxyacetate; methyl 3,7-dimethyl-2,6-octadienoate;

of acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;

of acyclic terpene aldehydes and ketones, such as, for example, geranial; neral; cirtonellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

of cyclic terpene alcohols, such as, for example, menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates, 3-methyl-2-butenoates thereof;

of cyclic terpene aldehydes and ketones, such as, for example, menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-iron; alpha-damascone; beta-damascone; beta-damascenone; delta-damascone; gamma-damascone; 1-(2,4,4-trimethyl-2-cyclo-hexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);

of cyclic alcohols, such as, for example, 4-tert-butylcyclohexanol; 3,3,5-trimethyl-cyclohexanol; 3-isocamphylcyclohexanol; 2,6,9-trimethyl-Z2,Z5,E9-cyclodode-catrien-1-ol; 2-isobutyl-4-methyltetrahydro-2H-pyran-4-ol;

of cycloaliphatic alcohols, such as, for example, alpha-3,3-trimethyl-cyclohexyl-methanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)-hexan-3-ol;

of cyclic and cycloaliphatic ethers, such as, for example, cineol; cedryl methyl ether; cyclododecyl methyl ether; (ethoxymethoxy)cyclo-dodecane; alpha-cedrene epoxide; 3a,6,6,9a-tetramethyldodecahydro-naphtho[2,1-b]furan; 3a-ethyl-6,6,9a-trimethyldodecahydro-naphtho[2,1-b]furan; 1,5,9-trimethyl-13-oxabicyclo[10,1,0]trideca-4,8-diene; rose oxide; 2-(2,4-dimethyl-3-cyclohexen-1-yl)-5-methyl-5-(1-methylpropyl)-1,3-dioxane;

of cyclic ketones, such as, for example, 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 5-cyclohexadecen-1-one; 8-cyclohexadecen-1-one; 9-cycloheptadecen-1-one; cyclopentadecanone;

of cycloaliphatic aldehydes, such as, for example, 2,4-dimethyl-3-cyclohexene-carbaldehyde; 2-methyl-4-(2,2,6-trimethyl-cyclohexen-1-yl)-2-butenal; 4-(4-hydroxy-4-methyl-pentyl)-3-cyclohexenecarbaldehyde; 4-(4-methyl-3-penten-1-yl)-3-cyclohexenecarbaldehyde;

of cycloaliphatic ketones, such as, for example, 1-(3,3-dimethyl-cyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl 2,4-dimethyl-3-cyclohexen-1-yl ketone;

of esters of cyclic alcohols such as, for example, 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;

of esters of cycloaliphatic carboxylic acids, such as, for example, allyl 3-cyclohexyl-propionate; allyl cyclohexyloxyacetate; methyl dihydrojasmonate; methyl jasmonate; methyl 2-hexyl-3-oxocyclopentane-carboxylate; ethyl 2-ethyl-6,6-dimethyl-2-cyclo-hexenecarboxylate; ethyl 2,3,6,6-tetramethyl-2-cyclohexenecarboxylate; ethyl 2-methyl-1,3-dioxolan-2-acetate;

of aromatic hydrocarbons, such as, for example, styrene and diphenylmethane;

of araliphatic alcohols, such as, for example, benzyl alcohol; 1-phenylethyl alcohol; 2-phenylethyl alcohol; 3-phenylpropanol; 2-phenyl-propanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

of esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate; of araliphatic ethers, such as, for example, 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

of aromatic and araliphatic aldehydes, such as, for example, benzaldehyde; phenyl-acetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butyl-phenyl)propanal; cinnamalde-hyde; alpha-butylcinnamaldehyde; alpha-amyl-cinnamaldehyde; alpha-hexylcinnamaldehyde; 3-methyl-5-phenylpentanal; 4-meth-oxybenzalde-hyde; 4-hydroxy-3-methoxybenzaldehyde; 4-hydroxy-3-ethoxybenz-aldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

of aromatic and araliphatic ketones, such as, for example, acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1 ,2,6-tetramethyl-3-(1-methyl-ethyl)-1H-5-indenyl]ethanone; 5′,6′,7′,8′-tetrahydro-3′,5′,5′,6′,8′,8′-hexamethyl-2-acetonaphthone;

of aromatic and araliphatic carboxylic acids and esters thereof, such as, for example, benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethyl-benzoate; ethyl 3-phenylglycidate; ethyl 3-methyl-3-phenylglycidate;

of nitrogen-containing aromatic compounds, such as, for example, 2,4,6-trinitro-1,3-dimethyl-5-tert-butylbenzene; 3,5-dinitro-2,6-dimethyl-4-tert-butylacetophenone; cinnamonitrile; 5-phenyl-3-methyl-2-pentenenitrile; 5-phenyl-3-methylpentane-nitrile; methyl anthranilate; methyl N-methylanthranilate; Schiff bases of methyl anthranilate with 7-hydroxy-3,7-dimethyloctanal; 2-methyl-3-(4-tert-butylphenyl)-propanal or 2,4-dimethyl-3-cyclohexenecarbaldehyde; 6-isopropylquinoline; 6-isobutylquinoline; 6-sec-butylquinoline; indole; skatole; 2-methoxy-3-isopropyl-pyrazine; 2-isobutyl-3-methoxypyrazine;

of phenols, phenyl ethers and phenyl esters, such as, for example, estragole; anethole; eugenole; eugenyl methyl ether; isoeugenole; isoeugenyl methyl ether; thymole; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

of heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

of lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexade-canolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The perfume oils comprising the compounds, according to the present invention, can be used for perfumings in liquid form, neat or diluted with a solvent. Suitable solvents for this purpose are, for example, ethanol, isopropanol, diethylene glycol monoethyl ether, glycerol, propylene glycol, 1,2-butylene glycol, dipropylene glycol, diethyl phthalate, triethyl citrate, isopropyl myristate and similar solvents known in the art.

In addition, the perfume oils comprising the compounds, according to the present invention, can be adsorbed on a carrier, which serves both to distribute the fragrances finely within the product and to release them in a controlled manner during use. Such carriers can be porous inorganic materials, such as light sulphate, silica gels, zeolites, gypsums, clays, clay granules, gas concrete or organic materials such as woods and cellulose-based substances.

The perfume oils comprising the compounds, according to the present invention, can also be microencapsulated, spray dried, in the form of inclusion complexes or in the form of extrusion products and are added in this form to the product to be perfumed.

The properties of the perfume oils modified in this way can optionally be further optimized by “coating” with suitable materials with regard to a more targeted fragrance release, for which purpose preference is given to using wax-like polymers such as, for example, polyvinyl alcohol.

The microencapsulation of the perfume oils can, for example, be carried out by the coacervation method using capsule materials made from, for example, polyurethane-like substances or soft gelatins. The spray-dried perfume oils can, for example, be prepared by spray drying an emulsion or dispersion comprising the perfume oil, where the carriers used can be modified starches, proteins, dextrin and vegetable gums. Inclusion complexes can be prepared, for example, by introducing dispersions of the perfume oil and cyclodextrins or urea derivatives into a suitable solvent, e.g., water. Extrusion products can be obtained by melting the perfume oils with a suitable wax-like substance and by extrusion with subsequent solidification, optionally in a suitable solvent, e.g. isopropanol.

In perfume compositions, the amount of compounds, according to the invention, used is from 0.05 to 50% by weight, preferably from 0.5 to 20%, based on the overall composition.

The perfume compositions according to the present invention, can be used in concentrated form, in solutions or in the above-described modified form for the preparation of fine perfumery products and industrial products.

Fine perfumery products and industrial products are, for example, perfume extracts, eau de parfum, eau de toilette, aftershave, eau de cologne, preshave products, splash colognes and perfumed freshening wipes, and the perfuming of acidic, alkaline and neutral cleaners, such as, for example, floor cleaners, window cleaners, dishwashing detergents, bath and sanitary cleaners, scouring milk, solid and liquid WC cleaners, pulverulent and foam carpet cleaners, liquid detergents, pulverulent detergents, laundry pretreatment agents such as bleaches, soaking agents and stain removers, fabric softeners, washing soaps, washing tablets, disinfectants, surface disinfectants, and of air fresheners in liquid or gel form or deposited on a solid carrier, aerosol sprays, waxes and polishes, such as furniture polishes, floor waxes, shoe creams, and bodycare compositions, such as, for example, solid and liquid soaps, shower gels, shampoos, shaving soaps, shaving foams, bath oils, cosmetic emulsions of the oil-in-water, water-in-oil and water-in-oil-in-water type, such as, for example, skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, haircare products, such as, for example, hair sprays, hair gels, hairsetting lotions, hair rinses, permanent and semipermanent hair colorants, hair-shaping compositions, such as cold waves and hair-smoothing compositions, hair tonics, hair creams and lotions, deodorants and antiperspirants, such as, for example, underarm sprays, roll-ons, deodorant sticks, deodorant creams, products for decorative cosmetics, such as, for example, eyeshadows, nail varnishes, make-up, lipsticks, mascara, and also candles, lamp oils, joss-sticks, insecticides, repellents and propellants.

For the perfuming of the products described, the amount of perfume composition can be from 0.1 to 40% by weight, preferably from 0.5 to 20% by weight, based on the end product.

The E- and/or Z-4,8-dimethyl-3,7-nonadien-2-one, according to the present invention, are prepared in a manner known per se.

4,8-Dimethyl-3,7-nonadien-2-one can advantageously be prepared from 4,8-dimethyl-3,7-nonadien-2-ol which has, for example, been synthesized by a Grignard reaction from citral and methylmagnesium chloride (Indian Perfum., 1983, 27, 112-18). The oxidation to give 4,8-dimethyl-3,7-nonadien-2-one is known (e.g., C. Aguilar et al. Bol. Inst. Quim. Univ. Nacl. Auton. Mex. 1969, 21, 226-240). This process uses reagents such as manganese dioxide and chromium trioxide, which are difficult to handle.

A preferred process for the preparation of 4,8-dimethyl-3,7-nonadien-2-one is characterized in that 4,8-dimethyl-3,7-nonadien-2-ol is reacted in the presence of a catalyst and a hydrogen acceptor.

The reaction can be illustrated by the following reaction equation:

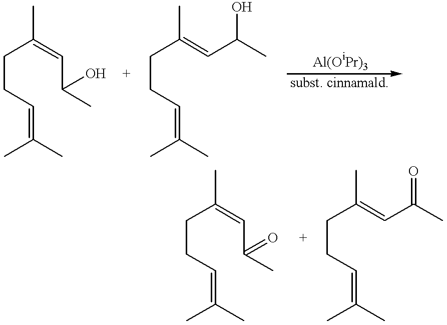

This reaction dispenses with critical reagents and is carried out without problems on a relatively large scale.

Catalysts which can be used are aluminum alkoxides, such as, aluminum tritertiary butoxide and aluminum triisopropoxide, preferably aluminum triisopropoxide.

The reaction temperature is between 40° C. and reflux temperature, preferably between 60-120° C.

As hydrogen acceptor, it is possible to use the known compounds (Org. Reactions, Vol. 6, pp. 207-272). It has, however, been found that the use of alpha-alkyl-substituted cinnamaldehyde derivatives, in particular, alpha-amyl and alpha-hexylcinnamaldehyde is preferred, since, in these cases, both the conversion is good and distillation is simplified. 4,8-Dimethyl-3,7-nonadien-2-one and the alkyl-substituted cinnamaldehyde and cinnamic alcohol derivatives can then be separated easily.

The present invention is further described in the following illustrative examples.

### EXAMPLES

### Example 1

Preparation of a perfume composition

The following are mixed (all data in grams):

TABLE 1

| | |
|---|---|
| Aldehyde C12 | 2 |
| Aldehyde C11 | 1 |
| Aldehyde C10 | 2 |
| Vertocitral (H & R) | 5 |
| Isoananate (H & R) | 5 |
| Dihydromyrcenol | 75 |
| Terpinyl acetate | 50 |
| Geranitrile (H & R) | 5 |
| Orange oil, white | 120 |
| Lavandin oil, Grosso | 20 |
| Spearmint oil, Americ. | 2 |
| Isobornyl acetate | 40 |
| Borneol L | 5 |
| Hydroxybenzylacetone, para | 1 |
| Lilial ® | 5 |
| Lyral ® | 10 |
| Linalool | 50 |
| Dimethylbenzylcarbinyl butyrate | 2 |
| Rose oxide, rac. | 1 |
| Damascone alpha 10% in DEP | 3 |
| Benzyl acetate | 10 |
| Jasmine absolve, synth. | 5 |
| Hexyl salicylate | 30 |
| Benzyl salicylate | 124 |
| Anethole | 2 |
| Coumarin | 5 |
| Cedrenyl acetate (IFF) | 15 |
| Patchouli oil | 15 |
| Piconia (IFF) | 10 |
| Sanel (H & R) | 5 |
| Evernyl (Givaudan) | 5 |
| Birch bark oil, 10% in DEP | 2 |
| Cuminaldehyde | 2 |
| Ambroxide 30% in Hercolyn 10% in DEP | 3 |
| Citral | 18 |

a) for use in an eau-de-toilette for men:

The addition of 20 g of 4,8-dimethyl-3,7-nonadien-2-one intensifies the freshness of the perfume and increases the impact.

b) for use in a deodorant roll-on:

The addition of 30 g of 4,8-dimethyl-3,7-nonadien-2-one intensifies the freshness effect, achieves a cosmetic-floral character and enhances the floral-salicylate-type components.

### Example 2

Preparation of a perfume composition

The following are mixed (all data in gram):

TABLE 2

| | |
|---|---|
| Aldehyde C12 | 2 |
| Aldehyde C11 | 2 |
| Aldehyde C10 | 3 |
| Aldehyde C8 | 5 |
| Galbanum resin | 10 |
| Bergamot oil, colorless | 150 |
| Terpinyl acetate | 50 |
| Geranitrile (H & R) | 3 |
| Lime oil terpene | 150 |
| Orange oil, white | 360 |
| Lilial ® | 5 |
| Geranium oil, synth. | 5 |
| Geraniol | 20 |
| Hexylcinnamaldehyde | 15 |
| Benzyl salicylate | 20 |
| Diethyl phthalate | 90 |
| 2,6-Di-tert.-butyl-4-methoxyphenol | 10 |

a) The addition of 20 g of 4,8-dimethyl-3,7-nonadien-2-one freshens and rounds off the base. The scent impression is long lasting. The concentration in a washing up liquid is 0.3%, in a shampoo is 0.5% and on an air freshener card is 50%.

b) The addition of 40 g of 4,8-dimethyl-3,7-nonadien-2-one gives a composition for the perfuming of white soap (concentration 1.2%). Compared with a base, which comprises citral instead of 4,8-dimethyl-3,7-nonadien-2-one, there were no discolorations of any kind.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Perfume compositions comprising E-4,8-dimethyl-3,7-nonadien-2-one and/or Z-4,8-dimethyl-3,7-nonadien-2-one of the formulae, 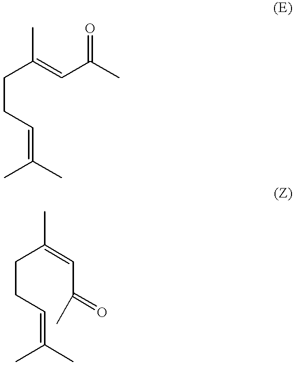
mixtures thereof and optionally in mixtures with further fragrances.

2. Perfume compositions according to claim 1, comprising E-4,8-dimethyl-3,7-nonadien-2-one and/or Z-4,8-dimethyl-3,7-nonadien-2-one and mixtures thereof in an amount of from 0.05 to 50% by weight, based on the total composition.

3. Perfume compositions according to claim 1, comprising E-4,8-dimethyl-3,7-nonadien-2-one and/or Z-4,8-dimethyl-3,7-nonadien-2-one and mixtures thereof in an amount of from 0.5 to 20% by weight, based on the composition.

4. A perfumery product comprising perfume compositions comprising E-4,8-dimethyl-3,7-nonadien-2-one and/or Z-4,8-dimethyl-3,7-nonadien-2-one, mixtures thereof and optionally in mixtures with further fragrances.

5. A perfumery product according to claim 4 wherein said perfume compositions are present in an amount of from 0.05 to 50% by weight, based on said perfumery product.

6. A perfumery product according to claim 5 wherein said perfume compositions are present in an amount of from 0.5 to 20% by weight, based on said perfumery product.

7. An industrial product comprising perfume compositions comprising E-4,8-dimethyl-3,7-nonadien-2-one and/or Z-4,8-dimethyl-3,7-nonadien-2-one, mixtures thereof and optionally in mixtures with further fragrances.

8. An industrial product according to claim 7 wherein said perfume compositions are present in an amount of from 0.05 to 50% by weight, based on the industrial product.

9. An industrial product according to claim 8 wherein said perfume compositions are present in an amount of from 0.5 to 20% by weight, based on the industrial product.
